(19) United States
(12) Patent Application Publication
(10) Pub. No.: US 2004/0209898 A1
(43) Pub. Date: Oct. 21, 2004

(54) Process for the preparation of risperidone

(75) Inventors: Reguri Buchi Reddy, Hyderabad (IN); Chakka Ramesh, Hyderabad (IN); Tamma Ranga Reddy, Hyderabad (IN); Kandirelli Venkata Kiran Kumar, Hyderabad (IN)

Correspondence Address: Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/761,803

(22) Filed: Jan. 21, 2004

(30) Foreign Application Priority Data
Jan. 21, 2003 (IN) 62/MAS/2003

(51) Int. Cl. A61K 31/519; C07D 487/04

(52) U.S. Cl. 514/259.41; 544/281

(57) ABSTRACT

The present invention is directed to a process for the preparation of the EPCRS form of Risperidone. The present invention also embodies a process for the preparation of EPCRS crystalline form of Risperidone which comprises, heating the Risperidone in an organic solvent(s) followed by subsequent cooling and isolation to get desired polymorph of Risperidone (Formula I).

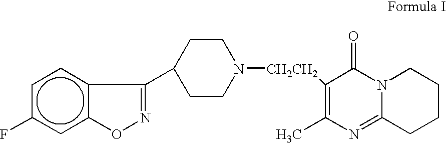

### CROSS-REFERENCE TO RELATED APPLICATIONS

This application claims the benefit of priority to Indian Patent Application No. 62/MAS/2003 filed Jan. 21, 2003, the disclosure of which is incorporated herein by reference in its entirety.

### FIELD OF INVENTION

The present invention relates to process for the preparation of crystalline form of 3-[2-[4-(6-Fluoro-1,2-benzisoxazol-3-yl)-1-piperidinyl]ethyl]-6,7,8,9-tetrahydro-2-methyl-4H-pyrido[1,2-a]pyrimidin-4-one. The product is also known as Risperidone in therapy and Risperidal as one of its familiar brand names. It may be represented by the following formula I.

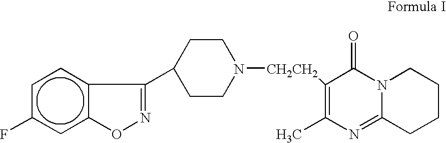

### BACKGROUND OF THE INVENTION

European patent EP 196132 B1 discloses certain 1,2-benzisoxazol-3-yl derivatives having psychotic and anti-serotonin activity including 3-[2-[4-(6-Fluoro-1,2-benzisoxazol-3-yl)-1-piperidinyl]ethyl]-6,7,8,9-tetrahydro-2-methyl-4H-pyrido [1,2-a]pyrimidin-4-one (Risperidone), which is a mixed 5-HT_{2A}/D₂-receptor antagonist and is an example of typical neuroleptic drug. The process for the preparation of Risperidone comprises condensation of two advanced intermediates i.e., 3-(2-chloroethyl)-6,7,8,9-tetrahydro-2-methyl-4H-pyrido[1,2-a]pyrimidine-4-one mono hydrochloride and 6-fluoro-3-(4-piperidinyl)-1,2-benzisoxazole in a solvent using a base.

The latest trend that has, of late, crept into the pharmaceutical industry is the studies on polymorphism in drugs and the difference in the activity of different polymorphic forms of a given drug. By the polymorphism we mean different physical forms, crystal forms, crystalline/liquid crystalline/non crystalline (amorphous) forms. This has especially become very interesting after observing that many antibiotics, antibacterials, tranquillisers etc. exhibit polymorphism and some/one of the polymorphic forms of a given drug exhibit superior bioavailability and consequently show much higher activity compared to other polymorphs.

The Summary Basis Of Approval (SBOA) submitted to the FDA by the inventors of Risperidone, disclose the existence of two polymorphic forms, polymorph 1 and polymorph 2 wherein polymorph 1 is stated as a more thermodynamically stable form, which is obtained by recrystallisation in Ethanol. However no substantial information is disclosed about polymorph 2 in prior art references.

The process disclosed in EP 196132 for preparation of pure Risperidone (Example 5), comprises recrystallisation of crude Risperidone in a mixture of dimethyl formamide and isopropyl alcohol to afford pure Risperidone.

US application 2002/0115673 and 2002/0115672 also discloses the process for the preparation of Risperidone including the process for the preparation of polymorphs Form-A, Form-B and Form-E. The process for the preparation of Risperidone comprises similar condensation as mentioned above but in their base forms i.e., 3-(2-chloroethyl)-6,7,8,9-tetrahydro-2-methyl-4H-pyrido[1,2-a]pyrimidine-4-one and 6-fluoro-3-(4-piperidinyl)-1,2-benzisoxazole using a solvent and a base.

X-ray powder diffractogram for EPCRS (European current reference standard) of Risperidone and X-ray powder diffractogram of the present invention are found to be similar indicating that the present invention is directed to prepare the EPCRS form of Risperidone.

The X-ray powder diffraction pattern of EPCRS of Risperidone is captured in the following table (table-1).

TABLE 1

| 2-Theta | Intensity |
|---|---|
| 7.137 | 2.5 |
| 10.751 | 4.4 |
| 11.544 | 8.1 |
| 14.343 | 37.5 |
| 14.964 | 13.3 |
| 15.546 | 4 |
| 16.510 | 9.3 |
| 18.652 | 12.6 |
| 18.992 | 22 |
| 19.923 | 17.1 |
| 21.386 | 100 |
| 22.236 | 4.1 |
| 22.541 | 11.1 |
| 23.300 | 25.1 |
| 23.620 | 6.7 |
| 24.556 | 2.1 |
| 25.403 | 3.8 |
| 27.619 | 3.9 |
| 28.663 | 3.7 |
| 29.077 | 14.7 |
| 32.574 | 2.3 |
| 33.260 | 3.5 |
| 38.642 | 2.1 |

The main objective of the present invention is to prepare the crystalline EPCRS form of 3-[2-[4-(6-Fluoro-1,2-benzisoxazol-3-yl)-1-piperidinyl]ethyl]-6,7,8,9-tetrahydro-2-methyl-4H-pyrido[1,2-a]pyrimidin-4-one (Risperidone), herein after termed as EPCRS for convenience.

### SUMMARY OF THE INVENTION

The present invention is directed to provide a process for the preparation of EPCRS form of Risperidone. The present invention also embodies a process for the preparation of EPCRS crystalline form of Risperidone which comprises, heating the Risperidone in an organic solvent(s) followed by subsequent cooling and isolation to get desired polymorph of Risperidone.

### BRIEF DESCRIPTION OF THE ACCOMPANYING DRAWINGS

FIG. 1 is X-ray powder diffractogram of EPCRS of Risperidone.

FIG. 2 is X-ray powder diffractogram of the present invention.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims. For purposes of the present invention, the following terms are defined below.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a powder, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds. A “compound” is a chemical substance that includes molecules of the same chemical structure regardless of its three dimensional orientation. Thus, it may be used to indicate racemates, stereoisomers, or both.

The term “pharmaceutical composition” is intended to encompass a product including the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

The term “isolating” is used to indicate separation of the compound being isolated regardless of the purity of the isolated compound from any unwanted substance, which is present with the compound as a mixture.

According to one aspect, the present invention provides a simple process for the preparation crystalline EPCRS form of Risperidone, which can be identified by X-ray powder diffraction as shown in Table-2.

TABLE 2

| 2-Theta | Intensity |
|---|---|
| 7.144 | 3.3 |
| 10.787 | 4.2 |
| 11.581 | 13.5 |
| 14.354 | 75.3 |
| 14.961 | 18.8 |
| 15.621 | 6.8 |
| 16.571 | 11.5 |
| 18.625 | 22 |
| 19.067 | 28.5 |
| 19.929 | 24.6 |
| 21.430 | 100 |
| 22.319 | 9.2 |
| 22.613 | 13.2 |
| 23.313 | 29.5 |
| 23.621 | 8.3 |
| 24.495 | 2.6 |
| 25.428 | 5.5 |
| 27.672 | 5.8 |
| 28.534 | 8.2 |
| 29.156 | 15.7 |
| 32.570 | 3.1 |
| 33.147 | 2.5 |
| 38.718 | 1.6 |

The significant d values obtained are 7.144±0.2, 10.787±0.2, 11.581±0.2, 13.84±0.2, 14.354±0.2, 14.961±0.2, 15.621±0.2, 16.571±0.2, 18.625±0.2, 19.067±0.2, 19.929±0.2, 21.430±0.2, 22.319±0.2, 22.613±0.2, 23.313±0.2, 23.621±0.2, 24.495±0.2, 25.428±0.2, 27.672±0.2, 28.534±0.2, 29.156±0.2, 32.570±0.2, 33.147±0.2 and 38.718±0.2.

The present invention also provides a simple process for the preparation of EPCRS form of Risperidone.

The crystalline EPCRS form of Risperidone of the present invention is prepared by a process, which comprises:

a) dissolving the Risperidone in an organic solvent(s) such as methyl propyl ketone, anisole, dioxane, methyl cellosolve, xylene, 1-pentanol, mixture of alcohols such as methanol or ethanol with solvents as acetone, methyl isobutyl ketone, methyl cellosolve, heptane, di-isopropyl ether, cyclohexane, isooctane, anisole, mixture of toluene with solvents such as acetone, iso octane, heptane, diisopropyl ether, mixture of xylene with solvents such as n-hexane, heptane, isooctane, t-butyl ether, mixture of methyl isobutyl ketone and methyl cellosolve, mixture of dichloromethane and iso octane, Mixture of methanol and water, Aqueous ethanol, mixture of chloroform and cyclohexane etc or a combination of above described solvents at hot condition or at reflux

b) optionally treating the dissolved solution with carbon

c) filtering the reaction solution to get particle free solution

d) cooling the reaction solution to get precipation/optionally adding the anti solvents such as n-hexane, n-heptane, isooctane, cyclohexane etc. for the separation of Risperidone from reaction solution.

e) isolating the desired EPCRS form of Risperidone by conventional methods.

The present invention hence is directed to the preparation of crystalline EPCRS form of Risperidone and also provides a simple and commercially viable process for its preparation. The following examples illustrate the invention but do not limit it in any way.

Risperidone can be prepared by the disclosed methods in EP 196132, US 2002/0115673A1 or US 2002/0115672A1.

### EXAMPLES

### Example 1

90.0 ml of Toluene was added to 10.0 g of Risperidone and the reaction solution was heated to reflux to obtain dissolution. The hot reaction solution was then treated with 1.0 g of carbon and filtered. The reaction solution was then slowly added to a flask containing 80.0 ml of iso octane at a temperature of 25-35° C. and stirred for 1-2 hours. The reaction solution was then filtered and the precipitate with washed with 10.0 ml of iso-octane and subsequently dried to afford Form-A of Risperidone (Yield 8.1 g, 81%).

### Example 2

15.0 ml of Methyl isobutyl ketone and 15.0 ml of methyl cellosolve was added to 5.0 g of Risperidone and the reaction solution was heated to reflux to obtain dissolution. The hot reaction solution was treated with 0.5 g of carbon and filtered. The reaction solution was then cooled to 0-5° C. and maintained at the same temperature for 1-2 hours. The separated solid was then filtered and washed with 5.0 ml of methyl isobutyl ketone. The resulted product was dried to afford Form-A of Risperidone (Yield 3.1 g, 62%).

### Example 3

30.0 ml of Methyl propyl ketone was added to 5.0 g of Risperidone and the reaction solution was then heated to reflux to obtain dissolution. The reaction solution was maintained at reflux for 5-20 minutes and then filtered. The reaction mass was then cooled to 25-35° C. and stirred at the same temperature for 1-2 hours and filtered. The precipitate was then washed with 5.0 ml of methyl propyl ketone and dried to afford Form-A of Risperidone (Yield 3.6 g, 72%).

### Example-4

25.0 ml of Xylene was added to 5.0 g of Risperidone and the reaction solution was heated to reflux to obtain dissolution. The reaction solution then maintained at reflux for 5-15 minutes and filtered. The reaction mass was then cooled to 25-35° C. and stirred at the same temperature for 1-2 hours and filtered. The precipitate was then washed with 5.0 ml of xylene and dried to afford Form-A of Risperidone (Yield 3.4 g, 68%).

### Example-5

25.0 ml of 1-Pentanol was added to 5.0 g of Risperidone and the reaction solution was heated to reflux and to obtain dissolution. The reaction solution was maintained at reflux for 15-30 minutes and filtered. The reaction mass was then cooled to 25-35° C. and stirred at the same temperature for 1-2 hours and filtered. The precipitate was then washed with 5.0 ml of 1-pentanol and dried to afford Form-A of Risperidone (Yield 3.4 g, 68%).

### Example-6

60.0 ml of 20% Aqueous ethanol was added to 10.0 g of Risperidone and the reaction solution was then heated to reflux to obtain dissolution. The hot reaction solution was then treated with 1.0 g of carbon and filtered. The reaction solution was then cooled to 0-5° C. and maintained at the same temperature for 30-60 minutes. The separated solid was then filtered and washed with 5.0 ml of 20% aqueous ethanol and dried to afford Form-A of Risperidone (Yield 5.3 g, 53%).

### Example 7

10.0 ml of Anisole and 10.0 ml of ethanol was added to 10.0 g of Risperidone and the reaction solution was then heated to reflux to obtain dissolution. The hot reaction solution was then treated with 11.0 g of carbon and filtered. The reaction solution was then cooled to 0-5° C. and maintained at the same temperature for 30-60 minutes. The separated solid and then filtered and washed with a mixture of 0.25 ml of anisole and 0.25 ml of ethanol and subsequently dried to afford Form-A of Risperidone (Yield 4.8 g, 48%).

### Example 8

7.8 ml of acetic acid was added to a reaction solution containing 50.0 ml of Water and 50.0 g of Risperidone. The resultant reaction solution was stirred for 5-10 minutes and the un-dissolved portion was filtered and washed with 25.0 ml of water. 200.0 ml of methanol was then added to the combined filtrate. The pH of the reaction mass was then adjusted to 8-9 with methanolic sodium hydroxide (6 g sodium hydroxide in 60 ml methanol). The suspension was stirred for about 30-60 minutes at a temperature of 20-35° C. and subsequently heated to reflux to obtain dissolution. The hot reaction solution was then treated with 5.0 g of carbon and filtered. The carbon bed was then washed with 10.0 ml of methanol. The filtrate was transferred into an another flask and stirred at 20-30° C. for 30-60 minutes, filtered and washed with 50.0 ml of water. The solid was then taken in 200 ml of water and stirred at 25-35° C. for 30-60 minutes, and subsequently filtered and washed with 50.0 ml of water and 50.0 ml of methanol and then dried to afford Form-A of Risperidone. (Yield 32.6 g, 65.2%).

The crystalline EPCRS polymorph of Risperidone obtained from above examples have similar XRD pattern in accordance with Figure (2).

### DETAILED DESCRIPTION OF THE ACCOMPANYING DRAWINGS

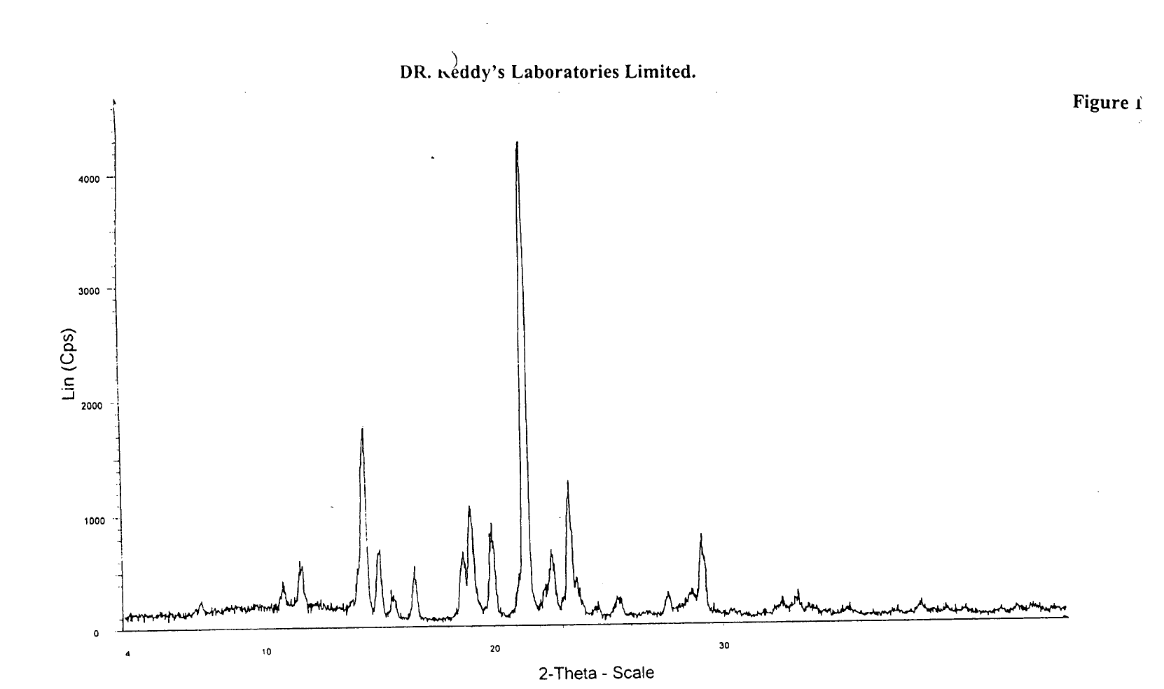

FIG. 1 is characteristic X-ray powder diffraction pattern of EPCRS of Risperidone. Vertical axis: Intensity (CPS); Horizontal axis: 2 Theta (degrees). The X-Ray diffraction pattern of EPCRS of Risperidone was measured on a Rigaku D/Max 2200 Powder Diffractometer with Cu Radiation source.

The significant two theta values obtained are 7.144 10.787, 11.581, 14.354, 14.961, 15.621, 16.571, 18.625, 19.067, 19.929, 21.340, 22.319, 22.613, 23.313, 23.621, 24.495, 25.428, 27.672, 28.534, 29.156, 32.570, 33.147 and 38.718.

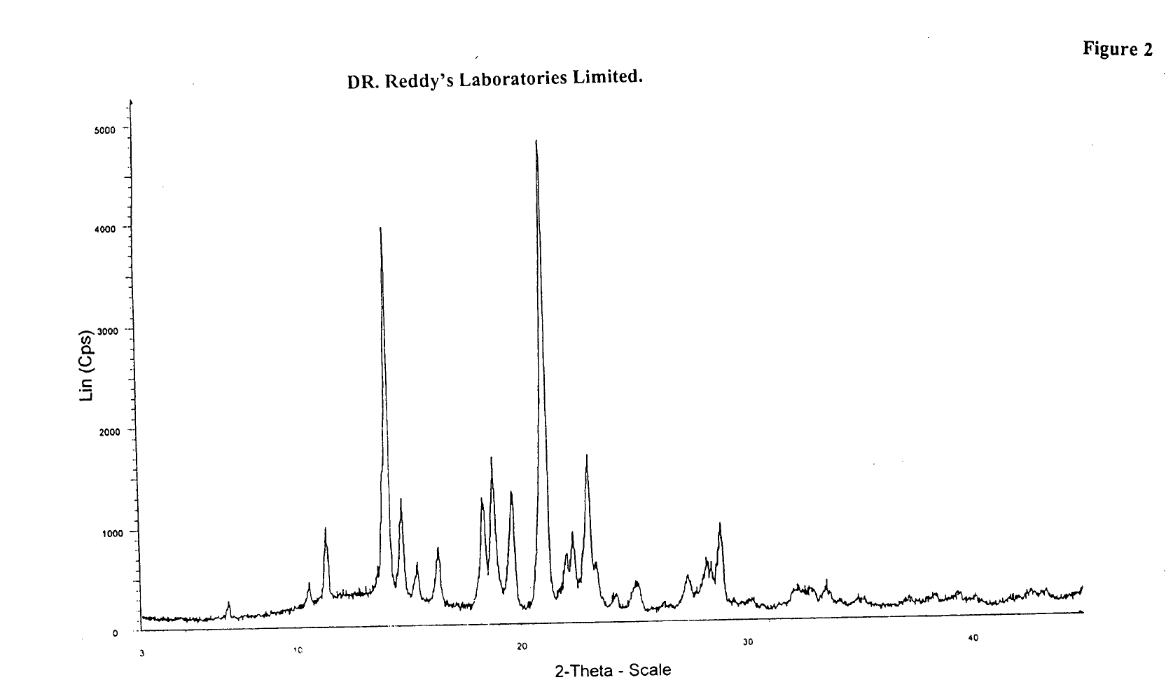

FIG. 2 is characteristic X-ray powder diffraction pattern of Polymorphic form of Risperidone obtained from the above examples. Vertical axis: Intensity (CPS); Horizontal axis: 2 Theta (degrees). The X-Ray diffraction pattern of novel polymorph-3 of Risperidone was measured on a Rigaku D/Max 2200 Powder Diffractometer with Cu Radiation source.

The significant d values obtained are 7.14±0.2, 10.79±0.2, 11.58±0.2, 13.84 ±0.2, 14.35±0.2, 14.96±0.2, 15.62±0.2, 16.57±0.2, 18.63±0.2, 19.07±0.2, 19.93±0.2, 21.43±0.2, 22.32±0.2, 22.61±0.2, 23.31±0.2, 23.62±0.2, 24.50±0.2, 25.43±0.2, 27.67 ±0.2, 28.53±0.2, 29.16±0.2, 32.57±0.2, 33.15±0.2 and 38.72±0.2.

We claim:

1. A process for the preparation of EPCRS form of 3-[2-[4-(6-Fluoro-1,2-benzisoxazol-3-yl)-1-piperidinyl]ethyl]-6,7,8,9-tetrahydro-2-methyl-4H-pyrido[1,2-a]pyrimidin-4-one (Risperidone), which comprises:
a) dissolving the Risperidone in an organic solvent(s) such as methyl propyl ketone, anisole, dioxane, methyl cellosolve, xylene, 1-pentanol, mixture of alcohols such as methanol or ethanol with solvents as acetone, methyl isobutyl ketone, methyl cellosolve, heptane, di-isopropyl ether, cyclohexane, isooctane, anisole, mixture of toluene with solvents such as acetone, iso octane, heptane, diisopropyl ether, mixture of xylene with solvents such as n-hexane, heptane, isooctane, t-butyl ether, mixture of methyl isobutyl ketone and methyl cellosolve, mixture of dichloromethane and iso octane, Mixture of methanol and water, Aqueous ethanol, mixture of chloroform and cyclohexane etc or a combination of above described solvents at hot condition or at reflux
b) optionally treating the dissolved solution with carbon
c) filtering the reaction solution to get particle free solution
d) cooling the reaction solution to get precipitation/optionally adding the anti solvents such as n-hexane, n-heptane, isooctane, cyclohexane etc. for the separation of Risperidone from reaction solution.
e) isolating the desired EPCRS form of Risperidone by conventional methods.

2. The process as claimed in claim 1 of step (a), dissolving the Risperidone in an organic solvent selected from methyl propyl ketone, anisole, dioxane, methyl cellosolve, methyl cellosolve, xylene and 1-pentanol.

3. The process as claimed in claim 1 of step (a), dissolving the Risperidone in a solvent mixture of alcohols such as methanol or ethanol with solvents like acetone, methyl isobutyl ketone, methyl cellosolve, n-heptane, di-isopropyl ether, cyclohexane, isooctane and anisole.

4. The process as claimed in claim 1 of step (a), dissolving the Risperidone in a solvent mixture of toluene or Xylene with solvents such as acetone, heptane, diisopropyl ether, n-hexane, isooctane and t-butyl ether.

5. The process as claimed in claim 1 of step (a), dissolving the Risperidone in a solvent mixture of methyl isobutyl ketone and methyl cellosolve

6. The process as claimed in claim 1 of step (a), dissolving the Risperidone in a solvent mixture of dichloromethane and isooctane

7. The process as claimed in claim 1 of step (a), dissolving the Risperidone in a solvent mixture of methanol and water.

8. The process as claimed in claim 1 of step (a), dissolving the Risperidone in Aqueous ethanol.

9. The process as claimed in claim 1 of step (a), dissolving the Risperidone in a mixture of chloroform and cyclohexane.

10. The process as claimed in claim 1 of step (a), dissolving the Risperidone in an Aqueous ethanol ranging 1% to 30%(water content in ethanol).

11. The process as claimed in claim 1 of step (a), dissolving the Risperidone in a solvent or solvents at hot condition ranging from 40° C. to reflux temperature.

12. The process as claimed in claim 1 of step (a), where in dissolving the Risperidone in a solvent or solvents mixture selected from any of the mentioned solvents in step (a) of claim 1.

13. The crystalline EPCRS form of Risperidone obtained in the process of claim 1 has X-ray powder diffraction pattern with peaks at 7.14±0.2, 10.79±0.2, 11.58±0.2, 13.84±0.2, 14.35±0.2, 14.96±0.2, 15.62±0.2, 16.57±0.2, 18.63±0.2, 19.07±0.2, 19.93±0.2, 21.43±0.2, 22.32±0.2, 22.61±0.2, 23.31±0.2, 23.62 ±0.2, 24.50±0.2, 25.43±0.2, 27.67±0.2, 28.53±0.2, 29.16±0.2, 32.57±0.2, 33.15±0.2 and 38.72±0.2 degrees two theta.
